(19) United States
(12) Patent Application Publication
(10) Pub. No.: US 2023/0190604 A1
(43) Pub. Date: Jun. 22, 2023

(54) DERMATOLOGIC/PERSONAL CARE CONCENTRATES

(71) Applicant: Vantage Specialty Ingredients, Inc., Warren, NJ (US)

(72) Inventors: Juan R Mateu, Ottawa, IL (US); Adam Perle, Miami, FL (US); Mackenzie Nunn, Newton, NJ (US); Adrianna R. Deremer, Wind Gap, PA (US)

(73) Assignee: Vantage Specialty Ingredients, Inc., Warren, NJ (US)

(21) Appl. No.: 18/111,196

(22) Filed: Feb. 17, 2023

Related U.S. Application Data
(63) Continuation-in-part of application No. 17/864,410, filed on Jul. 14, 2022, now Pat. No. 11,612,557.
(60) Provisional application No. 63/322,653, filed on Mar. 23, 2022.
(60) Provisional application No. 63/221,955, filed on Jul. 15, 2021.

(51) Int. Cl. A61K 8/365; A61K 8/67; A61K 8/34; A61K 8/73; A61Q 19/00; A61Q 5/00

(52) U.S. Cl. CPC A61K 8/365; A61K 8/676; A61K 8/345; A61K 8/735; A61K 8/733; A61Q 19/00; A61Q 5/002; A61K 2800/34; A61K 2800/33

(57) ABSTRACT

A substantially non-aqueous, stable, lyophilic dermatological concentrate comprised of: (i) a hydroxy acid, preferably selected from the group consisting of (a) azelaic acid at a concentration of at least about 20% or (b), ascorbic acid, at a concentration of up to about 30%; (ii) glycerin; and (ii) hyaluronan and/or an alginate, and derivatives thereof.

### CROSS REFERENCE TO RELATED APPLICATIONS

This application is a continuation-in-part of allowed U.S. non-provisional patent application Ser. No. 17/864,410. Priority is claimed under 35 U.S.C. § 119(e) to U.S. Provisional Application Ser. No. 63/322,653 (filed on Mar. 23, 2022). The disclosures of application Ser. No. 17/864,410 and 63/322,653 are incorporated herein by reference.

### FIELD OF INVENTION

Dermatologic and personal care products; methods of treating dermatologic conditions; methods for providing cosmetic benefits to skin (brighter and/or more even skin tone), reducing the appearance of facial fine lines/wrinkles, reducing the appearance of lesions and redness associated with inflammatory dermatoses including acne, rosacea and psoriasis, reducing the appearance of hair loss/thinning, and reducing skin and scalp oiliness.

### BACKGROUND OF THE INVENTION

Azelaic Acid is a dicarboxylic acid found in whole grain cereals—wheat, rye, barley—and is produced by Malassezia furfur, a commensal yeast fungus that is part of the human skin microbiome.

Azelaic acid is reported in the scientific literature to exhibit antibacterial, keratolytic, comedolytic, and antioxidant activity. It inhibits microbial cellular protein synthesis and reduces the thickness of the stratum corneum.

For treatment of acne vulgaris and inflammatory papules and pustules of mild to moderate rosacea, two prescription strengths are available in different dosage forms—20% active cream (Azelex®) and 15% active gel or foam (Finacea®). Lower “non-prescription” strength—10% active—preparations are marketed for cosmetic indications including as a brightener that reduces dullness, uneven tone, and textural irregularities.

One representative, non-prescription, skin brightening cream is The Ordinary® 10% Azelaic Acid Suspension. Other reported dermatologic benefits include reducing the production of sebum in the sebaceous gland, reducing hair loss or thinning, including by acting as a competitive inhibitor of the reduction of testosterone to dihydrotestosterone.

Finished products containing both azelaic acid and glycerin are known. Typically, azelaic acid is added to a thickened aqueous solution or gel. Illustrative is the formulation method described by Santoyo et al (1995), Int J Pharm, 117(2), 219-224. One gram Carbopol® powder is slowly added 100 ml water under constant stirring condition. The desired concentration of azelaic acid is added to this solution, which then swells (in about two hours), to obtain a homogeneous mixture. The mixture is stirred (e.g., for 60 minutes at 1,000 rpm), followed by addition of 0.5 ml of triethanolamine dropwise to adjust the pH. Benzoic acid and glycerin are added respectively as preservatives and humectants. Penetration enhancers (e.g., DMSO) can be added.

Another example of a dermatological preparation comprised of azelaic acid, glycerin as a humectant, and water is disclosed by Galderma as Example 2 in US Pre-Grant Patent Application Publication 2007/0219263.

U.S. Pat. No. 11,033,491 teaches a substantially non-aqueous, substantially alcohol-free foam comprised of azelaic acid and glycerin as a polar solvent that also functions as a humectant.

However, poor water solubility and permeability make incorporation of azelaic acid in topical products difficult. Bajaj, J.; Sharma D. (2015). Formulation and evaluation of topical azelaic acid gel. J. Chem. Pharm. Res., 7(10): 616-620.

U.S. Pat. No. 5,925,679, issued to Neutrogena and now expired, reports a solution to the formulation challenges of azelaic acid; and discloses a topical composition comprised of (i) 0.5%-10% wt/wt of “completely solubilized” azelaic acid having a pH of 4.0 or greater that is stable at “normal temperatures” in (ii) 20.0%-60.0% wt/wt of a “glycol base” selected from propylene glycol, polypropylene glycol, dipropylene glycol, butylene glycol, polyethylene glycol, polyethylene glycol ethers, polyproprylene glycol ethers, hexylene glycol, and ethoxydiglycol; and (iii) 20.0%-60.0% wt/wt of distilled water.

U.S. Pat. No. 6,734,210, expired, describes the difficulties of solubilizing azelaic acid. Water “only marginally dissolves” azelaic acid—to a maximum of about 0.24% by weight (w/w)—a concentration that is “not likely” to be clinically effective. Isopropanol is described as “good” solvent but “unsatisfactory” because it can be drying. Ethanol renders azelaic acid unstable at “normal temperatures” resulting in a “totally ineffective composition”. U.S. Pat. Nos. 4,713,394 and 4,885,282, similarly characterize the instability of azelaic acid when dissolved in ethyl alcohol.

Azeco Cosmeceuticals published a “Technical Information File” in Jul. 1, 2020 that presents guidance on formulating with azelaic acid and a generalized solution to the limited solubility of azelaic acid in aqueous systems—incorporating azelaic acid in a final formulation using a “premix”. According to Azeco, the most “straightforward” premix is created by dissolving azelaic acid in ethanol or isopropanol. However, Azeco notes that for many dermatologic preparations, these alcohols are not preferred; and, accordingly, diols are used—namely propanediol (available, for example, under the tradename Zemea® from DuPont Tate & Lyle Bio Products Company, LLC, Wilimington, Del.), 1,3-butylene glycol, and pentylene glycol (available, for example, under the tradename Pentiol Green+ from Minasolve SAS, Beuvry-La-Forêt, France). The Azeco formulation guidance also comments that “glycerin and glycerin/water mixtures are suitable solvent systems; [but] the maximum concentration[s] [of] water in this solvent mixture are [sic] not properly defined and not reported in the literature.”

Topical use of higher concentrations of azelaic acid—up to 20%—has been investigated. See, e.g., Graupe K, et al. “Efficacy and safety of topical azelaic acid (20 percent cream): An overview of results from European clinical trials and experimental reports.” Cutis. 1996;57(Suppl 1):20-35.

The efficacy of a 20% azelaic acid formulation in the treatment of hyperpigmentation has been favorably compared with hydroquinone. Balina L M, Graupe K. “The treatment of melasma. 20% azelaic acid versus 4% hydroquinone cream.” Int J Dermatol. 1991; 30:893-895

Lowe N J et al, “Azelaic Acid 20% Cream in the Treatment of Facial Hyperpigmentation in Darker-Skinned Patients.” Clin Ther. 1998; 20(5): 945-959;

Azelaic acid at concentrations of up to 20% has been effectively used in the treatment of acne.

Katsambas A. et al. “Clinical studies of 20% azelaic acid cream in the treatment of acne vulgaris: Comparison with vehicle and topical tretinoin.” Acta Derm-Venereol. 1989; 143:35-39;

Cavicchini S, Caputo R. “Long-term treatment of acne with 20% azelaic acid cream.” Acta Derm-Venereol Suppl. 1989; 143:40-44; Cunliffe W J, Holland K T. “Clinical and laboratory studies on treatment with 20% azelaic acid cream for acne.” Acta Derm- Venereol. 1989;143 :31-34.

Prior art azelaic acid creams have been criticized from an aesthetic/consumer use experience, notably for having a gritty feeling. See, e.g., https://thebeautyendeavor.com/naturium-azelaic-topical-acid-10-tranexamic-topical-acid-5/(“I love the idea of azelaic acid but I've always hated the thicker cream, often gritty texture.”)(accessed on Feb. 18, 2022).

The dermatologic/personal care concentrate of the present invention disclosure meets the need for a stable, dermatologic concentrate of azelaic acid or ascorbic acid that can be incorporated into topical formulations that have desirable cosmetic/aesthetic attributes, namely that are non-gritty in texture.

### SUMMARY OF THE INVENTION

A stable lyophilic dermatological concentrate that is substantially non-aqueous and substantially glycol-free and consists essentially of (i) a hydroxy acid, preferably selected from the group consisting of (a) azelaic acid at a concentration of at least about 20%, preferably at a concentration of at least about 25%, more preferably at a concentration of at least about 30%, even more preferably at a concentration of at least about 35%, still more preferably at a concentration of at least about 40%, still even more preferably at a concentration of at least about 45%; or (b) ascorbic acid, at a concentration of up to about 20%, preferably up to about 25%, more preferably at a concentration of up to about 30%, (ii) glycerin and (ii) hyaluronan and/or an alginate, and derivatives thereof.

### DETAILED DESCRIPTION

“Lyophilicity” refers to a colloidal system in which the dispersed phase has a high affinity for the dispersion medium. The colloidal nature of the system is confirmed by scattering of light.

As described below, stable, lyophilic dermatological concentrates of the present invention are prepared by combining (i) a high concentration of a hydroxy acid—namely, at a concentration of greater than 20% with (ii) glycerin and a salt of hyaluronan and/or an alginate.

“Hydroxy acid” means azelaic acid as well as an alpha-hydroxy acid (AHA), a polyhydroxy AHA, or a polycarboxylic AHA, each as defined below, and beta hydroxy acid.

“Alpha-hydroxy acid” (AHA) means a carboxylic acid with one hydroxyl group attached to the a-position of the carboxyl group and conform to the structure: (R₁)(R₂)C(OH)COOH, where R₁ and R₂ are selected from the group consisting of hydrogen, alkyl, aralkyl and aryl groups, the latter groups (alkyl, aralkyl and aryl) having from 1 to 29 carbon atoms. The alkyl, aralkyl and aryl groups may be saturated or unsaturated, isomeric or non-isomeric, straight or branched chain or cyclic. These groups may also contain as substituents OH, CHO, COOH and alkoxy groups having from 1 to 9 carbon atoms. R₁ and R₂ may be the same or different. In the latter case, the AHAs may be stereoisomers in the D, L, and DL forms. R₁ and R₂ may also be Cl, Br, I, S, F, or an alkyl or alkoxy group, saturated or unsaturated, having 1 to 9 carbon atoms. The term “AHA” is also to be understood to mean not only the free acid, but also its derivatives, including salts.

AHAs that may be included in the stable lyophilic dermatological concentrates of the present invention include: 2-hydroxyethanoic acid (glycolic acid, hydroxyacetic acid); 2-hydroxypropanoic acid (lactic acid); 2-methyl 2-hydroxypropanoic acid (methyllactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxynonanoic acid; 2-hydroxydecanoic acid; 2-hydroxyundecanoic acid; 2-hydroxydodecanoic acid (alpha hydroxylauric acid); 2-hydroxytetradecanoic acid (alpha hydroxymyristic acid); 2-hydroxyhexadecanoic acid (alpha hydroxypalmitic acid); 2-hydroxyoctadecanoic acid (alpha hydroxystearic acid); 2-hydroxyeicosanoic acid (alpha hydroxyarachidonic acid); 2-phenyl 2-hydroxyethanoic acid (mandelic acid); 2,2-diphenyl 2-hydroxyethanoic acid (benzilic acid); 3-phenyl 2-hydroxypropanoic acid (phenyl)acetic acid); 2-phenyl 2-methyl 2-hydroxyethanoic acid (atrolactic acid, 2-(4′-hydroxyphenyl); 2-hydroxyethanoic acid (4-hydroxymandelic acid); 2-(4′-chlorophenyl) 2-hydroxyethanoic acid (4-chloromandelic acid); 2-(3′-hydroxy-4′-methoxyphenyl) 2-hydroxyethanoic acid (3-hydroxy-4-methoxymandelic acid); 2-(4′-hydroxy-3′-methoxyphenyl); 2-hydroxyethanoic acid (4-hydroxy-3-methoxymandelic acid); 3-(2′-hydroxyphenyl); 2-hydroxypropanoic acid (3-(2′-hydroxy phenyl) lactic acid); 3-(4′-hydroxyphenyl) 2-hydroxypropanoic acid (3-(4′-hydroxyphenyl) lactic acid)); 2-(3′,4′-dihydroxyphenyl) 2-hydroxyethanoic acid (3,4-dihydroxymandelic acid).

“Polyhydroxy AHAs” that may be included in the stable lyophilic dermatological concentrates of the present invention include: 2,3-dihydroxypropanoic acid (glyceric acid); 2,3,4-trihydroxybutanoic acid and its isomers (erythronic acid, threonic acid); 2,3,4,5-tetrahydroxypentanoic acid and its isomers (ribonic acid, arabinoic acid, xylonic acid, lyxonic acid); 2,3,4,5,6-pentahydroxyhexanoic acid and its isomers (allonic acid, altronic acid, gluconic acid, mannoic acid, gulonic acid, idonic acid, galactonic acid, talonic acid); 2,3,4,5,6,7-hexahydroxyheptanoic acid and its isomers (glucoheptonic acid, galactoheptonic acid).

“Polycarboxylic AHAs” that may be included in the stable lyophilic dermatological concentrates of the present invention include: 2-hydroxypropane-1,3-dioic acid (tartronic acid); 2-hydroxybutane-1,4-dioic acid (malic acid); 2,3-dihydroxybutane-1,4-dioc acid (tartaric acid); 2-hydroxy-2-carboxypentane-1,5-dioic acid (citric acid); 2,3,4,5-tetrahydroxyhexane-1,6-dioic acid and its isomers (saccharic acid, mucic acid).

In one preferred embodiment, the hydroxy acid is salicylic acid, preferably in combination with cocamidopropyl dimethylamine. The preferred combination is commercially available from Vantage Specialty Ingredients, Inc. under the tradename Curcylic®.

Within the scope of the present invention disclosure HA includes hyaluronic acid and alkaline or alkaline-earth salts of hyaluronic acid, with sodium hyaluronate (NaHA) being a preferred salt of HA. NaHA may be of natural origin (e.g., extracted from rooster's combs) or fermented (derived from yeast and mold).

HA can be modified, where modification is via synthetic pathways known in the art including: reacting HA with adipic dihydrazide, with further crosslinking via acrylamide or hydrazone linkages; reacting HA with butane-1,4-diol diglycidyl ether; peroxidase crosslinking of HA with tyramide; formation of dialdehyde-modified HA by periodate oxidation; methacrylate modification of HA on the primary 6-hydroxyl group; esterification of HA (e.g., with benzyl ester); reaction of HA with glycidyl methacrylate; modification of carboxyl group of HA by reaction with 3,3′-di(thiopropionyl)bishydrazide (DTPH modification), followed by reduction of the disulfide bond with dithiothreitol; reaction of HA with bromoacetate; thiol-modified carboxymethyl HA can be crosslinked with thiol-modified gelatin using polyethylene (glycol) diacrylate.

Other methods of modification of HA known in the art, including as described in: Carbohyd Res. 2020 Mar; 489:107950; J Cosmet Dermatol. 2016 Dec;15(4):520-526.

High molecular weight hyaluronic acid (“HMW-HA”) means HA having a molecular weight greater than about 500 kDa, preferably greater than about 1,000 kDa, still more preferably from about 1,500 kDa to about 2,000 kDa.

“Non-HMW-HA” includes low molecular weight HA and medium molecular weight HA, each as defined below.

Low molecular weight hyaluronic acid (“LMW-HA”) means HA having a molecular weight of less than 25 kDa, preferably less than about 15 kDa, from about 1 kDa to 10kDa.

Medium molecular weight hyaluronic acid (“MMW-HA”) means HA having a molecular weight ranging from about 25 kDa to about 500 kDa.

In some embodiments, at least one HA is present in the stable, lyophilic dermatological concentrate of the present invention at a concentration of at least about 0.1%, preferably at a concentration of at least about 0.2%, more at a concentration of at least about 0.3%, still more preferably at a concentration of at least about 0.4%, and even more at a concentration of at least about 0.5%.

In other embodiments, at least one HA is present in the stable, lyophilic dermatological concentrate of the present invention at a concentration of at least about 0.75%, preferably at a concentration of at least about 1%, more preferably at a concentration of at least about 1.25%, still more preferably at a concentration of at least about 1.5%, and even more at a concentration of at least about 1.75%.

In still other embodiments, at least one HA is present in the stable, lyophilic dermatological concentrate at a concentration of at least about 2%, preferably at a concentration of at least about 5%, more preferably at a concentration of at least about 10%, even more preferably at a concentration of at least about 20%.

In some embodiments, the at least one HA is an HMW-HA.

In other embodiments, the at least one HA is an MMW-HA.

In still other embodiments, the at least one HA is an LMW-HA.

In certain embodiments, the ratio of HMW-HA to Non-HMW-HA is from about 4:1 to 1:4.

In other embodiments, the ratio of HMW-HA to Non-HMW-HA is about 2:1 to 1:2.

Alginates can be homopolymeric sequences of mannuronic acid (M blocks) homopolymeric sequences of guluronic acid (G blocks) and mixed sequences of mannuronic acid and glucuronic acid (MG blocks). Common algal sources of alginates include Laminaria digitata, Ecklonia maxima, Macrocystis pyrifera, Lessonia nigrescens, Ascophyllum nodosum, Laminaria japonica, Durvillea antartica, Durvillea potatorum and Laminaria hyperborea. As used in describing the present invention, “alginates” can include salts of alginic acid.

As described below, in certain embodiments, the stable, lyophilic dermatological concentrate may be prepared by adding and then water in a heating step.

In some embodiments the concentrates of the present invention may include ingredients that help reduce, remove or eliminate residual or associated or absorbed water.

In certain embodiments, a stable, lyophilic dermatological concentrate is prepared by combining an alginate and water to form a hydrated alginate, combining the hydrated alginate with glycerin, and mixing under heat—preferably a temperature of from about 40° C. to about 100° C., more preferably a temperature at least 90° C.—until the water content of the hydrated alginate/glycerin mixture is less than about 40%. Before heating, the alginate is present in the mixture with water and glycerin at a concentration of from about 0.5% to about 5%. After the heating step, the alginate comprises up to about 20%, preferably from about 3% to about 10%.

In other embodiments, at least one HA is added to the hydrated alginate/glycerin mixture before heating. In these embodiments, at least one HA may be present at a concentration ranging from about 0.1% to about 20% before heating, and from about 0.1% to about 2% after heating.

Because glycerin is hydrophilic, it can contain water in an associated or unfree or absorbed form and may absorb water from the atmosphere. The present invention disclosure is therefore directed to a “substantially non-aqueous” concentrate.

A first novel and basic property of the stable lyophilic dermatological concentrate of the present invention disclosure is that the concentrate is “substantially non-aqueous” or “substantially waterless”, by which is meant the inventive concentrate has a water content below about 5%, preferably below about 2%, more preferably below about 1%, still more preferably below 0.5%; even more preferably below 0.1%; and most preferably no detectable water content.

In a first set of embodiments, the present invention disclosure is directed to a stable lyophilic dermatological concentrate consisting essentially of at least about 20% azelaic acid, preferably at least about 25%, more preferably at least about 30%, even more preferably at least about 35%, azelaic acid, and still more preferably at least about 40%; and a salt of hyaluronan and/or an alginate. In certain embodiments, azelaic acid preferably comprises up to about 45% and even more preferably at least about 50% of the stable lyophilic dermatological concentrate.

In this first set of embodiments, the stable lyophilic dermatological concentrate consists essentially of less than about 80% of glycerin, preferably less than about 75%, more preferably at less than about 70%, even more preferably less than about 65%, and still more preferably less than about 60%.

In a second set of embodiments, the stable lyophilic dermatological concentrate is comprised of, consists essentially of, or consists of at least about 20% ascorbic acid, more preferably at least about 25%, even more preferably at least about 30%; and a salt of hyaluronan and/or an alginate.

In this second set of embodiments, the stable lyophilic dermatological concentrate of the present invention disclosure is comprised of, consists essentially of, or consists of less than about 80% of glycerin, preferably less than about 75%, more preferably at less than about 70%.

A second novel and basic property of the stable lyophilic dermatological concentrate of the present invention disclosure is that the concentrate is “substantially glycol-free” by which is meant the concentrate contains less than about 5%, preferably below about 2%, more preferably below about 1%, still more preferably below 0.5%, even more preferably below 0.1%, and most preferably none of the following glycols: propylene glycol, butylene glycol, pentylene glycol.

In certain embodiments, a third novel and basic property of the stable lyophilic dermatological concentrate of the present invention disclosure is that the concentrate is “substantially alcohol-free” by which is meant the concentrate contains less than about 5%, preferably below about 2%, more preferably below about 1%, still more preferably below 0.5%, even more preferably below 0.1%, and most preferably none of the following short-chain alcohols: ethanol, propanol, isopropanol, butaneol, iso-butaneol, t-butaneol and pentanol.

By “stable” is meant no visible separation or settling of particles after: (i) three freeze/thaw cycles; (ii) storage at 50° C. for one month; and, in preferred embodiments, (iii) after three months at 40° C., 25° C. (room temperature) and 5° C. (refrigerated).

An essential characteristic of the stable lyophilic dermatological concentrate of the present invention disclosure is that the concentrate is substantially free of “grit”—both in terms of appearance and feel.

Preferably, the stable lyophilic dermatological concentrate of the present invention disclosure is substantially anhydrous.

In certain embodiments, the stable lyophilic dermatological concentrate of the present invention disclosure may be made by a two-step process: in first step, water is combined with azelaic acid and glycerin; and, thereafter, water is removed by heating, thereby creating a substantially non-aqueous and substantially glycol-free dermatological concentrate that consists essentially of at least about 20% azelaic acid or up to about 30% ascorbic acid.

In these embodiments, without addition of water, combining glycerin and the hydroxy acid (azelaic acid or ascorbic acid) will result in a blend in which some particles may be visible, and grit felt. Surprisingly and unexpectedly, mixing a high concentration of azelaic acid (>20%) with glycerin in the presence of water as described above, followed by heating the mixture to a temperature sufficient to remove water and create a substantially non-aqueous concentrate (having less than about 5%, preferably less than about 2%, more preferably below about 1%, still more preferably below 0.5%; even more preferably below 0.1%; and most preferably no detectable water content), results in a blend in which particles are substantially imperceptible, visually and by touch.

Without wishing to be bound by a theory, it is believed that the substantial absence of particles/crystals of azelaic acid or ascorbic acid—particles/crystals that are substantially perceptible, visually and by touch—is attributable to addition of water, which promotes hydrogen bonding of the hydroxy acid. Addition of water followed by dehydration results in lyophilicity; and, therefore, improved solubility and higher concentration of the hydroxy acid in glycerin. In contrast, a concentrate made with glycerin alone (without addition water) allows for some hydrogen bonding but not enough to solubilize an optimized high concentration of azelaic acid in a manner that achieves little to no perceptible grit.

The stable lyophilic dermatological concentrate of the present invention disclosure can include one or more waxes, which can be either hydrogenated (fully or partially) or non-hydrogenated natural waxes, such as those obtained from animal, botanical, or mineral sources, or synthetic waxes. Some synthetic waxes are synthesized using one or more components from natural sources.

Natural waxes that are animal waxes include beeswax, lanolin, shellac wax, and whale wax.

Natural waxes that are botanical waxes include candelilla wax, castor wax, cotton wax, soy wax, jojoba wax, olive wax, carnauba wax, sugar cane wax, rice bran wax, bayberry wax, sunflower wax, rose petal wax, and Japan wax. Sunflower wax is a preferred botanical wax.

Mineral waxes include montan wax, ozokerite, and ceresin.

Petroleum-based waxes include paraffin wax and microcrystalline wax.

Synthetic waxes include polyethylene waxes (e.g. Jeenate® waxes available from Jeen International of Fairfield N.J.), silicone waxes, fluoro waxes, Fischer-Tropsch waxes, polypropylene waxes, esters of poly(ethylene glycol), and pegylated sorbitans, alone or in combination with, for example, monoalkyl ethers of poly(ethylene glycol) (e.g. ceteareth-20).

Synthetic waxes also include so-called “functionalized waxes, a non-limiting example of which are pegylated animal waxes (e.g., PEG-8 beeswax)

In certain embodiments, the wax is a synthetic emulsifying wax, for example glyceryl monostearate, to mention just one. Self-emulsifying wax, as that term is used herein, refers to a chemically modified wax that contains at least one emulsifier component, e.g., a non-ionic emulsifier.

The stable lyophilic dermatological concentrate of the present invention disclosure can include one or more cosmetically-acceptable oils or esters. Preferred esters and oils include jojoba oil and meadowfoam seed oil.

The stable lyophilic dermatological concentrate of the present invention disclosure can include one or more “active ingredients” that is/are soluble in glycerin, at a concentration that provides one or more skin benefits. Active ingredients may be added to the concentrate at the following levels from at least about 0.01% on a weight/weight basis based on the weight of the concentrate, preferably at least about 0.1%, more preferably at least about 0.5%, and still more preferably at least about 1.0%. Non-limiting examples of active ingredients include: agents for the treatment of an inflammatory dermatosis, including acne, psoriasis or rosacea; anti-microbial and anti-fungal actives; anti-itch agents; topical anaesthetics; emollients and skin soothing agents; non-steroidal anti-inflammatory agents; humectants and moisturizing agents, including hyaluronic acid and its derivatives; antioxidants and agents that reduce the appearance of fine lines and wrinkles, including vitamins, proteins and peptides; skin bleaching and lightening agents; and plant extracts.

Non-limiting examples of surfactants that can be added to the stable lyophilic dermatological concentrate of the present invention include: Cocoamidopropyl Betaine; Sodium Cocoyl Isethionate; Sodium Lauroamphoacetate; Sodium Methyl Cocoyl Taurate; Sodium Lauryl Sulfate (SLS); Sodium Laureth Sulfate (SLES). In certain preferred embodiments, the surfactant is neither SLS nor SLES. Surfactants can be cationic (e.g., Stearamidopropyl Dimethylamine).

Emulsifiers that can be included in the stable lyophilic dermatological concentrate include: oil-in-water emulsifiers (e.g., Ceteareth-20); silicone-in-water emulsifiers (e.g., PEG-12 Dimethicone); water-in-oil emulsifiers (e.g., Polyglyceryl-4 Oleate); water-in-silicone emulsifiers (e.g., PEG/PPG-30/10 Dimethicone or Lauryl PEG/PPG-18/18 Methicone).

Emulsifiers that can be included in the stable lyophilic dermatological concentrate may be cationic, anionic, non-ionic, or amphoteric.

Two non-limiting but preferred examples of multi-functional, cationic, conditioning emulsifiers that can be included in the Lyophilic Colloid are Behentrimonium Methosulfate, Behentriumonium Chloride, Stearamidopropyl Dimethylamine, or Cetrimonium Chloride.

Along with surfactant(s)/emulsifier(s), one or more oils can be included in the stable lyophilic dermatological concentrate.

The stable lyophilic dermatological concentrate can be added to a finished product at concentrations ranging from 1% to 80%, preferably from about 5% to 50%, and more preferably from about 10% to about 60%. By “finished product” is meant a formulation (also known in the art as a “preparation”) that can be applied directly to a keratinous substrate (skin, scalp/hair). The finished product can be in the form a cream, lotion, gel, or serum.

In one set of preferred embodiments, the stable lyophilic dermatological concentrate containing the hydroxy acid, preferably azelaic acid or ascorbic acid, is poured into a mold forming a stick.

The stable lyophilic dermatological concentrate containing the hydroxy acid, preferably azelaic acid or ascorbic acid, can be spray congealed or poured and cast into a block, flaked, or prilled, and, optionally, ground to smaller particle size and/or passed through a sieve to achieve a desired particle size cut-off.

### EXAMPLES

The following examples are illustrative. Modifications will be apparent to, and can be readily made by, those skilled in the art without departing from the spirit and scope of the invention. The scope of the appended claims is not to be limited to the examples.

| | | Sodium | | | | |
|---|---|---|---|---|---|---|
| | | Hyaluro- | Sodium | Azelaic | Ascorbic | |
| Example | Glycerin | nate | Alginate | Acid | Acid | Water |
| 1 | QS | 0.2 | 0.0 | 30 | 0.0 | 0.0 |
| 2 | QS | 0.0 | 0.2 | 40 | 0.0 | 0.0 |
| 3 | QS | 0.5 | 0.0 | 0.0 | 10 | 0.0 |
| 4 | QS | 0.0 | 0.5 | 0.0 | 15 | 0.0 |
| 5 | QS | 0.2 | 0.0 | 30 | 0.0 | 0.1 |
| 6 | QS | 0.0 | 0.2 | 40 | 0.0 | 0.1 |
| 7 | QS | 0.5 | 0.0 | 0.0 | 20 | 0.1 |
| 8 | QS | 0.0 | 0.5 | 0.0 | 20 | 0.1 |

### Example 9

The substantially non-aqueous, stable, dermatologic lyophilic concentrate of Example 1 is prepared by further addition of 0.9 grams of Polyglyceryl 4 Oleate, 8.2 grams of Glycol Stearate and 9.1 grams of Sweet Almond Oil.

### Example 10

A stable dermatologic lyophilic concentrate is prepared by combining 10 grams of water, 40 grams of azelaic acid and 20 grams of glycerin. The mixture is heated to a temperature sufficient to remove water and create a substantially anhydrous concentrate.

1. A substantially non-aqueous, stable, lyophilic dermatological concentrate comprised of: (i) a hydroxy acid, preferably selected from the group consisting of (a) azelaic acid at a concentration of at least about 20% or (b), ascorbic acid, at a concentration of up to about 30%; (ii) glycerin; and (ii) hyaluronan and/or an alginate, and derivatives thereof.

2. The substantially non-aqueous, stable, lyophilic dermatological concentrate of claim 1 wherein the hydroxy acid is ascorbic acid at a concentration of up to 25%.

3. The substantially non-aqueous, stable, lyophilic dermatological concentrate of claim 2 wherein ascorbic acid at a concentration of up to 20%.

4. The substantially non-aqueous, stable, lyophilic dermatological concentrate of claim 1 wherein the hydroxy acid is azelaic acid at a concentration of at least 20%.

5. The substantially non-aqueous, stable, lyophilic dermatological concentrate of claim 4 wherein azelaic acid at a concentration of at least 25%.

6. The substantially non-aqueous, stable, lyophilic dermatological concentrate of claim 5 wherein azelaic acid at a concentration of at least 30%.

7. The substantially non-aqueous, stable, lyophilic dermatological concentrate of claim 6 wherein azelaic acid at a concentration of at least 35%.

8. The substantially non-aqueous, stable, lyophilic dermatological concentrate of claim 7 wherein azelaic acid at a concentration of at least 40%.

9. The substantially non-aqueous, stable, lyophilic dermatological concentrate of claim 8 wherein azelaic acid at a concentration of at least 45%.

10. A substantially non-aqueous, stable, lyophilic dermatological concentrate made by a process of
(a) creating a mixture of (i) glycerin, (ii) a hydroxy acid selected from the group consisting of azelaic acid at a concentration of at least 20% and ascorbic acid at a concentration of up to 30%, and (iii) hyaluronan and/or an alginate, and derivatives thereof, and (iv) water; and
(b) heating the mixture from step (a) to a temperature sufficient to remove water and create a substantially non-aqueous, stable concentrate
wherein azelaic acid or ascorbic acid particles are substantially imperceptible in appearance and touch.

11. The substantially non-aqueous, stable, lyophilic dermatological concentrate of claim 10 having less than about 2% water.

12. The substantially non-aqueous, stable, lyophilic dermatological concentrate of claim 13 having less than about 1% water.

13. The substantially non-aqueous, stable, lyophilic dermatological concentrate of any of claim 11 that is substantially glycol-free.

14. A substantially non-aqueous, stable, lyophilic dermatological concentrate made by a process of
(a) creating a mixture of (i) glycerin, (ii) a hydroxy acid selected from the group consisting of azelaic acid at a concentration of at least 20% and ascorbic acid at a concentration of up to 30%, and (iii) hyaluronan and/or an alginate, and derivatives thereof;
(b) adding water to the step (a) mixture; and
(b) heating the mixture from step (a) to a temperature sufficient to remove water and create a substantially non-aqueous, stable concentrate
wherein azelaic acid or ascorbic acid particles are substantially imperceptible in appearance and touch.

15. A finished formulation including the stable lyophilic dermatological concentrate of claim 1.

16. The finished formulation of claim 15 further comprising one or more surfactants and/or one or more oils or esters.

17. The finished formulation of claim 15 that is surfactant free.

18. The finished formulation of claim 15 that does not contain any oils.

19. The finished formulation of claim 15 that does not contain any silicones.
